(19) United States
(12) Patent Application Publication
(10) Pub. No.: US 2022/0178808 A1
(43) Pub. Date: Jun. 9, 2022

(54) PROCESS AND APPARATUS FOR QUANTIFYING SOLID RESIDUE ON A SUBSTRATE

(71) Applicant: THE CHEMOURS COMPANY FC, LLC, WILMINGTON, DE (US)

(72) Inventors: SCOTT C. BROWN, HOCKESSIN, DE (US); DANIEL C. KRAITER, WILMINGTON, DE (US); PETER JERNAKOFF, WILMINGTON, DE (US); CARLOS ALEXIS VELEZ, SMYRNA, DE (US); MICHAEL PATRICK DIEBOLD, WILMINGTON, DE (US)

(73) Assignee: THE CHEMOURS COMPANY FC, LLC, WILMINGTON, DE (US)

(21) Appl. No.: 17/435,411

(22) PCT Filed: Feb. 26, 2020
(86) PCT No.: PCT/US2020/019885

§ 371 Date: Sep. 1, 2021

Related U.S. Application Data
(60) Provisional application No. 62/812,543, filed on Mar. 1, 2019.
(60) Provisional application No. 62/933,712, filed on Nov. 11, 2019.

(51) Int. Cl. G01N 15/06

(52) U.S. Cl. CPC G01N 15/0606; G01N 2033/0096

(57) ABSTRACT

The present invention relates to a process and apparatus for quantifying solid residue on a sample. The process includes using a solid substrate and an aerosolizing device, adding a solid material to the aerosolizing device, forming a particle cloud of solid particles, wherein at least 1% of the mass concentration of solid particles have a mass median aerodynamic particle diameter up to about 10 μm, thus applying the solid particles to the solid substrate(s) to form treated substrate(s), maintaining at a temperature of from about 30 to about 120° C. for at least a portion of the process, and removing a portion of solid particles from the treated substrate(s), and analyzing said at least one sample. The present invention further comprises an apparatus for applying solid particles to a substrate. The process can be used, for example, to analyze the dirt pickup resistance of a solid sample.

### FIELD OF THE INVENTION

A solid material is aerosolized and applied to at least one substrate, which substrate is then treated and analyzed for solid residue.

### BACKGROUND OF THE INVENTION

Surfaces exposed to environmental conditions such as dirt, dust, rust, and pollution can collect solid residue over time that is difficult or costly to replace or remove. This is especially true of exterior surfaces, such as painted or sided buildings, exposed to outdoor conditions. Testing surfaces that have been exposed to these real conditions takes many months or years for proper data collection, and because each location has different environmental conditions, testing can require a large amount of resources. Although a number of processes have been developed to analyze the amount or effect of residue on such surfaces, it has been difficult to find an accelerated method that correlates directly to real-world exposure data.

A number of methods have been used to apply a particulate solid to a substrate. Solids have been applied by brush (Li et al., “Dependence of Dirt Resistance of Steel Topcoats on Their Surface Characteristics”, J. Coat. Technol. Res., 10 (3) 339-346, 2013), or by casting an aqueous slurry (Khanjani et al., “Improving Dirt Pickup Resistance in Waterborne Coatings Using Latex Blends of Acrylic/PDMS Polymers”, Progress in Organic Coatings, 102 (2017) 151-166; Zhou et al., “A Novel Adsorption Method to Simulate the Dirt Pickup Performance of Organic Coatings”, J. Coat. Technol. Res., 15 (1) 175-184, 2018). However, these application methods do not accurately simulate the natural particle deposition process.

### BRIEF SUMMARY OF THE INVENTION

The need exists for an accelerated process of quantifying solid particle adsorption that correlates to analysis of substrates treated by long-time real-world exposure. Also desirable is an apparatus for applying the solid particles to one or more substrates. The present invention meets these needs.

The present invention relates to a process for quantifying solid residue on a sample comprising: 1) providing at least one solid substrate and an aerosolizing device having an inlet and an outlet, 2) adding a solid material to the inlet, 3) forming a particle cloud of solid particles, wherein at least 1% of the mass concentration of solid particles have a mass median aerodynamic particle diameter up to about 10 μm, the particle cloud of solid particles exiting the aerosolizing device through the outlet, thus applying said solid particles to said at least one solid substrate to form at least one treated substrate, 4) wherein said at least one treated substrate is maintained at a temperature of from about 30 to about 120° C. for at least a portion of the process, 5) removing a portion of said solid particles from said at least one treated substrate, where steps 4) and 5) are performed in any order to form at least one sample, and 6) analyzing said at least one sample.

The present invention further comprises an apparatus comprising a) an enclosure, b) an aerosolizing device comprising a lumen extended from an inlet at one end to an outlet at another end, wherein the lumen is in fluid communication with the enclosure, and wherein the lumen allows an aerosol stream comprising gas and solid material to flow through the aerosolizing device and to exit the outlet of the aerosolizing device, c) a port on the enclosure for adding solid material to the aerosolizing device, and d) at least one solid substrate located in the enclosure, wherein the aerosolizing device further comprises: a particle dispersion unit for reducing agglomerates and/or aggregates to solid particles wherein at least 1% of the mass concentration of solid particles have a mass median aerodynamic particle diameter up to about 10 μm, wherein said at least one solid substrate is located inside the enclosure and positioned to avoid direct contact with the aerosol stream exiting the outlet of the aerosolizing device.

### BRIEF DESCRIPTION OF THE DRAWINGS

FIG. 1 is a side view of an apparatus of the invention, with arrows indicating gas or aerosol stream flow direction.

FIG. 2 is a cross section view of an apparatus of the invention, with arrows indicating gas or aerosol stream flow direction.

FIG. 3 is a cross section view of the aerosolizing device, with arrows indicating gas or solid material flow.

FIG. 4 shows a paint film coated panel showing direction of paint film application and location of cut solid substrates.

FIG. 5 shows a cut solid substrate showing label placement.

FIG. 6 shows a cut solid substrate showing placement of attachment mechanism and untreated paint film surface before heat treatment.

FIG. 7 shows a treated solid substrate showing ambient temperature solid particle treatment.

FIG. 8 shows a treated solid substrate showing solid particle treatment after heat treatment.

FIG. 9 shows a treated solid substrate showing four separate areas: Area 1 (untreated paint film surface after heat treatment); Area 2 (ambient temperature solid particle treatment after double tape peel); Area 3 (solid particle treated film after heat treatment then double tape peel); and Area 4 (solid particle treated film after heat treatment and without solid particle removal).

FIG. 10 is a laser diffraction particle analyzer graph showing particle size distribution.

FIG. 11 depicts images of disassembled aerosol sampling collection devices, SKC PM2.5 before introduction of a solid powder (FIG. 11A), SKC PM2.5 after introduction of a Flamrus 101 solid powder (FIG. 11B), and SKC PM10 after introduction of a Flamrus 101 solid powder (FIG. 11C).

### DETAILED DESCRIPTION OF THE INVENTION

Trademarks are indicated herein by capitalization.

The present invention provides a process for quantifying solid residue on a sample and an apparatus for applying solid particulates to a sample. The process allows for reliable accelerated testing of one or more treated substrates. Also, because a variety of solid particle compositions and post-treatment conditions may be applied, the process can mimic a variety of environments, climates, and locations. The apparatus applies solid particles in an aerosolized form, which more closely resembles environmental pollutants and conditions.

The present invention relates to a process for quantifying solid residue on a sample comprising: 1) providing at least one solid substrate and an aerosolizing device having an inlet and an outlet, 2) adding a solid material to the inlet, 3) forming a particle cloud of solid particles wherein at least 1% of the mass concentration of solid particles have a mass median aerodynamic particle diameter (MMAD) up to about 10 μm, the particle cloud of solid particles exiting the aerosolizing device through the outlet, thus applying said solid particles to said at least one solid substrate to form at least one treated substrate, 4) wherein said at least one treated substrate is maintained at a temperature of from about 30 to about 120° C. for at least a portion of the process, 5) removing a portion of said solid particles from said at least one treated substrate, where steps 4) and 5) are performed in any order to form at least one sample, and 6) analyzing said at least one sample.

The present invention further comprises an apparatus comprising a) an enclosure, b) an aerosolizing device comprising a lumen extended from an inlet at one end to an outlet at another end, wherein the lumen is in fluid communication with the enclosure, and wherein the lumen allows an aerosol stream comprising gas and solid material to flow through the aerosolizing device and to exit the outlet of the aerosolizing device, c) a port on the enclosure for adding solid material to the aerosolizing device, and d) at least one solid substrate located in the enclosure, wherein the aerosolizing device further comprises: a particle dispersion unit for reducing agglomerates and/or aggregates to solid particles, wherein at least 1% of the mass concentration of solid particles have a MMAD up to about 10 μm, wherein said at least one solid substrate is located inside the enclosure and positioned to avoid direct contact with the aerosol stream exiting the outlet of the aerosolizing device.

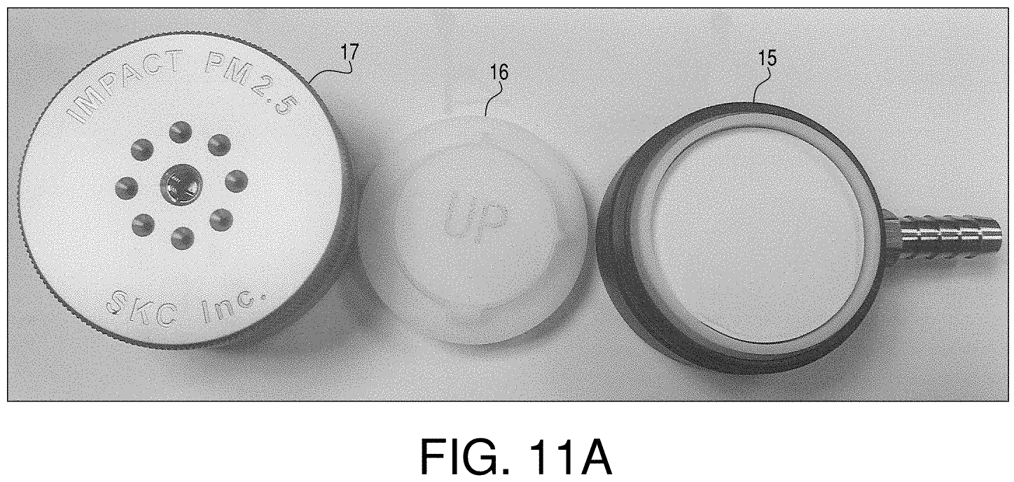

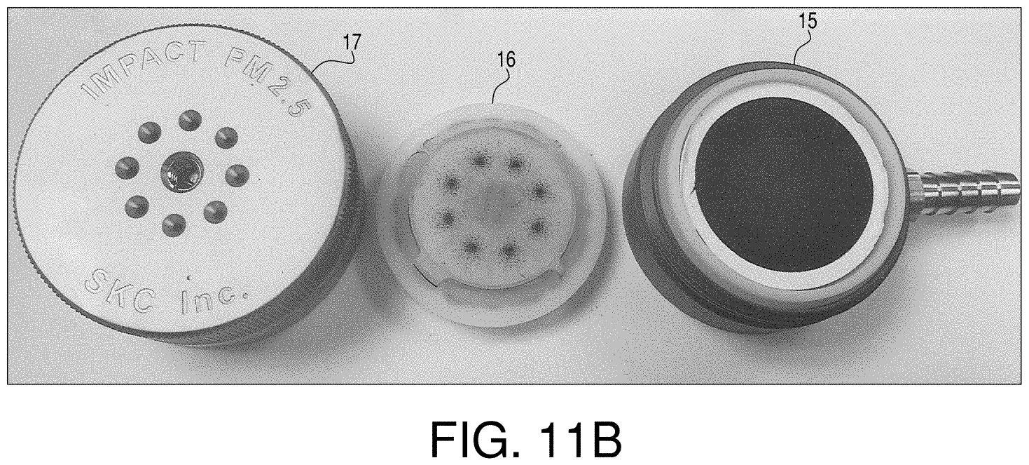

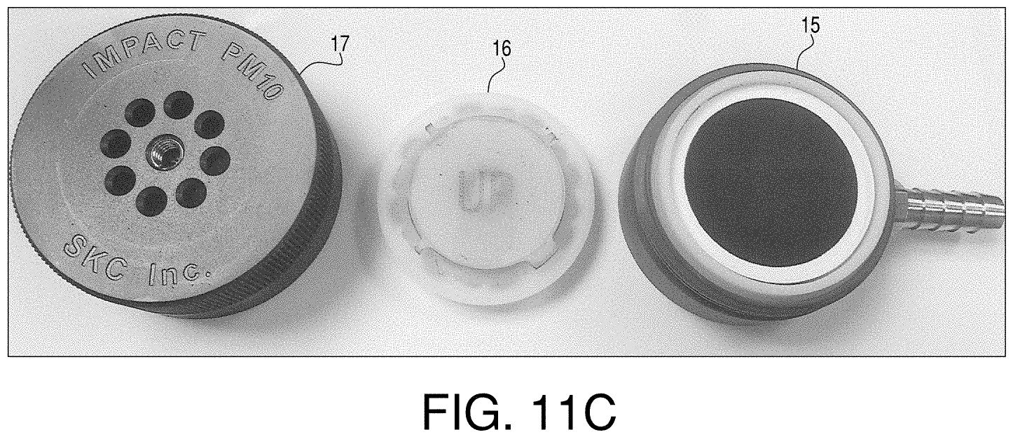

The term “reducing agglomerates and/or aggregates to solid particles” is intended to cover the process of overcoming cohesive van der Waals and capillary forces of a bulk powder or solid material in its natural state. A solid powder material, which is inherently agglomerated and/or aggregated in its natural state, is added to the aerosolizing device, at which point the solid material is broken down by applied energy to form individual particles, or into smaller agglomerates and/or aggregates. The present invention also relates to a process as above, where the solid particles have a mass concentration of particles up to about 10 μm in MMAD of more than 1% as determined by a US Federal Reference Standard 40 CFR Part 50. For example, an aerosol sampling collection device such as a PM10 aerosol sampling collection device may be used. When MMAD values are expected to be below 2.5 μm, a PM2.5 aerosol sampling collection device may be used. The mass concentration of particles having a MMAD of up to about 10 μm, or up to about 2.5 μm, is determined by the formula:

$$\frac{\begin{matrix}mass collected in an aerosol \\ sampling collection material\end{matrix}}{total mass of particles sampled} \times 100 ,$$

where the aerosol sampling collection device corresponds to the targeted maximum MMAD. The total mass of the particles sampled is the sum of the masses of particles entering the sampling collection device during the sampling period. This total mass of particles sampled is determined by measuring the mass increase of the entire device after sampling or by the summation of the deposited mass on impaction surfaces plus the aerosol sampling collection material (e.g., PM10 and/or PM2.5 content). For example, in the disassembled aerosol sampling collection devices of FIG. 11, the “mass collected in an aerosol sampling collection material” corresponds to the mass increase of quartz filter 15 after sampling. The “total mass of particles sampled” corresponds to the total mass increase of quartz filter 15, impaction disc 16, and filter cassette casing 17. Suitable PM10 and TM2.5 devices comply with US Federal Reference Standard 40 CFR Part 50. A compendium of suitable measurement devices is maintained by the US EPA Ambient Air Monitoring Technology Center.

The invention can be understood with reference to the Figures. According to the method, in step 1) an aerosolizing device 1 having an inlet 2 and outlet 3 are provided, along with at least one solid substrate 12. In step 2), a solid material is added to an aerosolizing device 1 through the inlet 2, such as through port 4. The port 4 may be in any shape, and it may take any form, such as a simple particle dosing port or opening, a tube or pipe of varied shape including a J-shape, a tube or pipe having a control valve, or a dosing device. The aerosolizing device 1 comprises a lumen extended from an inlet 2 at one end to an outlet 3 at another end, wherein the lumen is in fluid communication with the enclosure 9. The lumen may have any suitable shape or form, for example cylindrical, cuboid, conical, pyramidal, etc.

In one aspect, no liquid carriers or components are used when adding the solid material to the aerosolizing device 1. The solid material may be any material that is desired to quantify. It may be any material of contrasting color, in relation to said at least one substrate, that retains its particle form under the temperature, pressure, and moisture conditions of the process. Examples of solid materials include but are not limited to carbon black, iron oxide, graphite, ash, soot, crushed brick dust, dirt, pollen, spores, inorganic crystallites, or mixtures thereof. Ash may include coal ash, rice-straw ash, modified rice-straw ash such as methyltrimethoxysilane-modified rice-straw ash, or mixtures thereof.

The aerosolizing device 1 is connected to the enclosure 9 such that the outlet 3 of the aerosolizing device 1 is in fluid communication with the enclosure 9. In one embodiment, the outlet 3 of the aerosolizing device extends into the enclosure. Although shown as a cylindrical shape, enclosure 9 and aerosolizing device 1 can be any suitable shape or form, for example cylindrical, cuboid, conical, pyramidal, etc. The solid material flows through the aerosolizing device 1, which includes a particle dispersion unit 5 with a particle dispersion zone 6. By particle dispersion unit 5, it is meant a unit that disperses and/or separates agglomerates and/or aggregates of solid material into individual particles or into smaller agglomerates and/or aggregates. Once the solid material reaches the particle dispersion zone 6, the agglomerates and/or aggregates of solid material are broken into solid particles having a mass median aerodynamic particle diameter up to about 10 μm. This serves to perform step 3) of the inventive process, which describes forming a particle cloud of solid particles, wherein at least 1% of the mass concentration of solid particles have a MMAD up to about 10 μm. The solid particles may also have a Peclet number up to about 1. Naturally occurring dirt, dust, and pollutants are distributed in the air as small particles. In a test process, the size and distribution of the solid particles are critical, so they can accurately represent solid particles in a particular outdoor or indoor environment. The aerodynamic particle diameter can be defined as the diameter of a sphere with density 1000 kg/m³ that has the same sedimentation velocity in quiescent air as the test particle. The Peclet number describes the balance between gravitational forces promoting sedimentation and thermal motion facilitating surface force mediated interactions. It is herein defined by the mathematical equation:

$$P e = \frac{2 π Δ ρ g {a}^{4}}{3 k T}$$

where Pe is the Peclet number, Δρ=density of the particle (ρ_{particle})−bulk density of the solid material (ρ_{bulk}), g is the acceleration of gravity (9.8 m/s²), a is the spherical equivalent particle radius, k is Boltzman's constant (1.38×10⁻²³ J/K), and T is temperature in Kelvin. “Spherical equivalent particle radius” is defined as the radius of a spherical particle with an equivalent settling velocity or mobility. Peclet number (Pe) is determined for the purpose of this embodiment by measuring the particle size through the use of laser diffraction conformant to ISO TC24/SC4 TS 13320 and using the obtained laser diffraction mean volume particle size as the “spherical equivalent” particle radius. The density of the powder is determined to be the packed bulk density of the powder as measured by ASTM D7481-18 divided by 0.64.

For dust particles to avoid rapid sedimentation they must have low average aerodynamic particle diameters or Pe numbers and therefore exist as small particles and clusters of low inertia. Bulk powders, on the other hand, settle rapidly and flow as large particles or clusters governed by inertia. Bulk powders have Pe numbers on the order of 100, or approximate sizes above 50 μm, or above 100 μm, and exist as coagulates, agglomerates, or aggregates. In the bulk, agglomerated state, the solid material no longer acts as individual particles but instead as a particle cluster. To simulate the physical interactions of natural dust particles, or other particles of a particular environment, the agglomerates and/or aggregates must be broken down into solid particles of lower Pe numbers. In one aspect, the solid particles have aerodynamic particle diameters of about 10 nm to about 20 μm. In another aspect, the solid particles have an aerodynamic particle diameter of about 100 nm to about 10 μm; and in a third aspect, the solid particles have an aerodynamic particle diameter of about 200 nm to about 2.5 μm. In one aspect, at least about 1% to about 100% of the mass concentration of solid particles have a MMAD up to about 10 μm; in another aspect, at least about 1% to about 100% of the mass concentration of solid particles have a MMAD up to about 5 μm; and in a third aspect, at least about 1% to about 100% of the mass concentration of solid particles have a MMAD up to about 2.5 μm. In one aspect, at least about 10% to about 100% of the mass concentration of solid particles have a MMAD up to about 10 μm; in another aspect, at least about 10% to about 100% of the mass concentration of solid particles have a MMAD up to about 5 μm; and in another aspect, at least about 10% to about 100% of the mass concentration of solid particles have a MMAD up to about 10 μm.

A number of mechanisms may be used as the particle dispersion unit to reduce the solid material to a solid particle. In one aspect, a carrier gas is introduced to the aerosolizing device at intake 7. The carrier gas flows to a chamber within the aerosolizing device and is forced through one or more ports 8 of the particle dispersion unit at the particle dispersion zone. In one aspect, the carrier gas is pressurized to create a gas stream of high velocity, meaning the step of adding a solid material to the aerosolizing device 1 further comprises adding a carrier gas. The gas can be pressurized to any pressure necessary, or heated or cooled to any temperature necessary, to achieve the above-noted MMAD or Peclet number. Examples of gas composition include air, nitrogen, argon, carbon dioxide, oxygen, water vapor, or mixtures thereof. The change in pressure (ΔP) between the intake 7 and particle dispersion zone 6, defined as Pᵢₙₜₐₖₑ−P_{dispersion zone}, can be 0.1 to 200 psi. In one aspect, ΔP is 1 to 100 psi, and in another aspect, ΔP is 5 to 60 psi. Pressure for each region can be measured by an air pressure gauge.

When the gas enters the particle dispersion zone 6, it mixes and collides with the solid material to disperse the material into an aerosol stream having solid particles and gas. The particle dispersion unit 5 may be an eductor, such as a modified eductor having a venturi design or having high intensity nozzles, or it may be an exhaustive eductor, a slurry eductor, an evacuating eductor, or jet eductor. Alternatively, the aerosolization device may be a rotating brush apparatus, a rotating drum, a vortex shaker, a fluidized bed, a nebulizer, or a slurry atomizer. The aerosol stream exits the outlet 3 of the aerosolizing device 1. In one aspect, the aerosolizing device 1 forces the aerosol stream through the lumen at a velocity up to about 50 m/s. In another aspect, the aerosolizing device 1 forces the aerosol stream through the lumen at a velocity up to about 16 m/s. In a third aspect, the aerosolizing device 1 forces the aerosol stream through the lumen at a velocity up to about 5 m/s. Velocity can be measured by a calibrated heated wire anemometer.

The aerosol stream then enters the enclosure 9 and forms a particle cloud that may contact said at least one solid substrate 12. The aerosolizing device 1 may be positioned such that the flow is in any direction. For example, the aerosolizing device may be positioned such that the aerosol stream flows downward, upward, horizontally, or at an angle from horizontal. The enclosure 9 may further contain one or more flow diverters 10, where the flow diverter 10 is positioned in the path of the aerosol stream exiting the aerosolizing device 1 to divert the aerosol stream away from the solid substrate(s) 12. In one aspect, the flow diverter 10 is below the aerosolizing device 1 and forces the aerosol stream upward. In another aspect, the flow diverter 10 is above the aerosolizing device 1 and forces the aerosol stream downward. In one aspect, the aerosol stream contacts a surface from a frame of the enclosure 9 to divert the aerosol stream away from the solid substrate(s) 12.

The apparatus may further contain a housing 11 having an open end inside the enclosure, which housing partially or completely surrounds the outlet of the aerosolizing device 1. Although shown as a cylindrical shape, housing 11 can be any suitable shape or form. Housing 11 may direct the flow of the aerosol stream away from part or all of the frame of the enclosure 9 and from the solid substrate(s) 12. The apparatus may further comprise one or more openings 13 on the enclosure that connects the contents of the enclosure to atmospheric pressure, vacuum, a pressurized area, or a means for recirculating solid material. The one or more openings 13 can be any suitable shape or form, for example, circular, square, etc. The apparatus may also contain one or more exhaust ports 14 to allow gas to escape. The one or more exhaust ports 14 can be any suitable shape or form, for example, circular, square, etc.

In one aspect, the step of applying said particles to at least one solid substrate is performed by positioning the at least one solid substrate to avoid direct contact with the outlet of the aerosolizing device and allowing the particle cloud to contact the at least one solid substrate. This can be done by positioning the solid substrate outside of the housing 11 and away from the open end of said device, or between the flow diverter and the frame of the enclosure away from the aerosol stream.

One or multiple substrates may be used simultaneously in the process and apparatus of the invention. The at least one solid substrate may be any substrate that is typically in contact with solid particles. Examples include but are not limited to plastic, wood, wood and/or paper laminate, a solid surface having a coating such as polymeric, wood, wood laminate, paper laminate, or a solid surface having a coating, wherein the coating is a polymer coating, non-polymeric organic coating, or inorganic coating polymer coating, non-polymeric organic coating, ceramic coating, or inorganic coating. Examples of a polymer coating include a pigmented or unpigmented paint coating, clear coating, adhesive coating, or composite coating. For example, a paint chip or painted panel, a vinyl siding sample, a laminated panel, or a plastic film may be used. The at least one solid substrate may be held in place by any attachment mechanism, provided there is enough exposed surface area for testing, including but not limited to adhesive; adhesive tape; brackets; a hook and loop mechanism such as Velcro™ a ball and stick mechanism such as 3M Command™ strips; a holder designed for the substrate to slide into a slot; or magnets. The at least one solid substrate may be exposed to the particle cloud for any amount of time suitable for the test. For example, the at least one solid substrate is exposed to the particle cloud until the change in color of said substrate measured in CIE L*a*b* color space is five times greater than the color measuring device detection limit. Color can be measured by using a colorimeter, a light spectrophotometer, optical microscopy or digital imaging and image analysis. In one aspect, the at least one solid substrate is exposed to the particle cloud for 0.1 to 60 minutes. In another aspect, the at least one solid substrate is exposed to the particle cloud for 0.5 to 20 minutes. In one aspect, said at least one solid substrate is exposed to the particle cloud once; in another aspect, one or more substrates are exposed to the particle cloud multiple times in different sessions.

Although not necessary, methods to increase the amount of solid particles contacting the solid substrate or methods to decrease the amount of time to contact solid particles to the one or more solid substrates may be used. Examples include applying electrostatic energy, thermophoretics, field focusing, rotational force, high speed mixing, continuous drop, pressure change, or aerodynamic enclosure design. For that reason, the apparatus may further comprise an electrostatic charging unit, a test sample cooling apparatus, a flow diverter, additional aerosol generation devices including a rotating brush generator, dispersion atomization, laser abrasion, sudden vacuum release, rotating drum mechanism, vortex mechanism, high speed mixer, or continuous drop mechanism.

In step 4), at least one treated substrate is maintained at a temperature from about 30 to about 120° C. for at least a portion of the process. This treatment step is intended to simulate outdoor conditions or warm indoor environments. If the environment to be tested typically has a low temperature, it is also suitable to expose the treated substrate to lower temperatures. At elevated temperatures, treated substrates that include components that flow, such as polymer components in a coating or in the substrate body itself, may adsorb solid particles. Thus, the desired temperature depends on the environment to be simulated as well as the flow or other characteristics of the substrate. A rigid substrate whose morphology and properties are similar at elevated temperatures and non-elevated temperatures may not require heat treatment. In one aspect, the at least one treated substrate is maintained at a temperature from about 40 to about 80° C., and in another aspect, the treated substrate is maintained at a temperature from about 40 to about 60° C. Although the temperature is maintained for at least a portion of the process, this can be for any desired time period. For example, at least one substrate is maintained at the desired temperature for 5 minutes to 1 month; in another aspect, at least one substrate is maintained at the desired temperature for 1 hour to 14 days; and in another aspect, at least one substrate is maintained at the desired temperature for 1 hour to 3 days. This step may be performed by placing the treated substrates in an oven or other controlled elevated temperature environment; heating an enclosure containing the treated substrates and the aerosolizing device; absorption of light; convective heating; conductive heating; or applying directed heat, such as with a forced air dryer, direct contact with a heated liquid, heated gas, or solid heated element, or applying radiant heat. This step may also include exposing the treated substrate to humidity to simulate environmental humidity or to liquid water to simulate rain, rinsing, or pressure-washing.

The treated substrate(s) may contain solid particles embedded in the substrate(s) as well as solid particles that are removable from the surface of the treated substrate(s) that have been exposed to treatment. The process of this invention is used to quantify the amount of solid particles that are not readily removable from the treated substrate(s). For this reason, step 5) requires removing a portion of said solid particles from the treated substrate(s). The particles that are not embedded in the substrate will be removed in this step. The step of removing the solid particles can be performed by contacting the at least one sample with an adhesive tape or tacky surface and removing the tape or tacky surface, contacting with and removing a silicone film, applying vacuum, mechanical wiping, liquid washing, rubbing, or the use of a liquid or air jet. In one aspect, the step of removing the solid particles can be performed by contacting the at least one sample with one of the above-mentioned methods for a short period of time, for example less than 5 minutes, less than 1 minute, or less than 30 seconds.

If adhesive tape is used, the adhesive should be selected such that no residue is left on the treated substrate(s) after contact. The adhesive from the adhesive tape is selected such that it will cleanly remove at least some solid particles but will not remove a coating or surface of the treated substrate. In another aspect, a test adhesive or removal method is determined to be capable if it can be employed to remove test particles deposited on a standard microscope slide in one or more steps. The test adhesive is suitable if it does not alter the test substrate with respect to the color measurement method. Suitability is determined by measuring the color of the intended test material, applying the particle removal method to an unaltered test material surface then remeasuring the color. Suitable methods do not induce a color change greater than five times the detectable color change for the method. The same may be said of the contact with a tacky surface or with a silicone film. Examples of adhesive tape include adhesives capable of removing weakly attached particles, such as Scotch® Magic Tape™ (3M, MN) pressure sensitive adhesive tape or similar. Scotch® Magic Tape™ 810 has a synthetic acrylic adhesive of approximately 22 micrometers in thickness and adhesion to steel of approximately 2.5 N/cm per ASTM D-3330. In one aspect, the adhesive tape or film has an adhesion to steel of about 0.1 N/cm to 100 N/cm. In another aspect, the adhesive tape or film has an adhesion to steel of about 0.5 N/cm to 50 N/cm; and in a third aspect, the adhesive tape or film has an adhesion to steel of about 1 N/cm to 40 N/cm. Other useful adhesive tapes include, but are not limited to, no-residue duct tape such as 3M NO RESIDUE Duct Tape (3M, MN), poster tape such as Scotch Removable Poster Tape (3M, MN), UltraTape 7155 (UltraTape, OR), painter's tape such as FrogTape Painter's Tape (Shurtech, OR), or packaging tape such as Duck Brand EZ Start Packaging Tape (Shurtech, OR).

Steps 4) and 5) may occur in any order. In one aspect, the heating step 4) occurs before solid particle removal step 5). In another aspect, the solid particle removal step 5) occurs before heating step 4). In one aspect, the process contains an additional step 1a) of exposing at least one solid substrate to a temperature of about 4 to about 120° C. before application of solid particles in step 3). In one aspect, step 1a) is performed at a temperature from about 10 to about 80° C., and in another aspect, the step 1a) is performed at a temperature from about 40 to about 60° C. Other additional steps may also be used. For example, simulated exposure to different media or conditions may be achieved by further treating at least one solid substrate prior to solid particle application step 3). Water or humidity may be applied to the substrate to simulate natural exposure to elements including, but not limited to, environmental debris, humidity, rain, rinsing, or pressure-washing.

The sample is then analyzed for effects of solid particle deposition. For example, the sample may be analyzed for mass or weight, brightness, color, reflectance, or chemical composition changes. Such characteristics may be analyzed using a balance, colorimeter, or an Fourier Transform Infrared Spectroscopy (FTIR) instrument. In one aspect, the solid substrate is analyzed prior to application of solid particles in step 3), and the result is compared to the result of treated substrate after step 5). The process may be used to treat a solid substrate once, or it may be repeated on the same solid substrate to demonstrate repeated exposure.

Success of the process and apparatus is demonstrated by comparing real-world samples to a sample of the invention.

### Test Methods and Materials

All solvents and reagents, unless otherwise indicated, were purchased from Sigma-Aldrich, St. Louis, Mo., and used directly as supplied.

7 in 1 Future Color, Shield-1 Nano Semi-Gloss, Shield-1 Nano Sheen, Supershield Semi-Gloss, and Supershield Sheen are paints commercially available from TOA Paint, Thailand.

Aquis Façade and Novasil are paints commercially available from Tikkurila OYJ, Finland.

Natrosol 250 MHR is commercially available from Ashland Chemicals, Columbus, Ohio

Tamol 165A, Kathon LX, Rhoplex VSR 1049 LOE, Rhopaque Ultra, Acrysol RM2020 NPR, and Acrysol SCT-275 are commercially available from Dow Chemical, Philadelphia, Pa. Propylene glycol is commercially available from Dow Chemical Canada, Calgary, AB.

BYK-348 is commercially available from BYK Chemie, Wallingford, Conn.

Foamstar ST2434 is available from BASF, Florham Park, N.J.

Ti-Pure™ R-706 and Ti-Pure™ Select TS-6300 are TiO₂ products available from The Chemours Company, Wilmington, Del.

Minex 4 is commercially available from The Cary Company, Canada Nephon, ON.

Diafil 525 is commercially available from Celite, Lompoc, Calif.

Texanol is commercially available from Eastman Chemicals, Kingsport, Tenn.

Ammonia is available from EMD Millipore Corporation, Billerica, Mass.

Flamrus 101 is a carbon black powder obtained from Degussa AG, Germany.

Lamp Black 101 Powder is a carbon black powder available from Orion Engineered Carbons S.A., Luxembourg.

The following test methods and materials were used in the examples herein.

### Test Methods

### Preparation A. Preparation of Paint Film Coated Panels for Accelerated and Outdoor Exposure Testing

Control and experimental paints were drawn down by hand on 30.48 cm long×10.16 cm wide×0.06 cm thick aluminum panels (Q-Lab: Westlake, Ohio) using a slightly modified, 0.10 mm gap clearance, stainless steel bar film applicator (Byk-Gardner, Columbia, Md.) in conjunction with a stainless steel vacuum plate (Paul M. Gardner Co: Pompano Beach, Fla.). Said modification involved the application of a single layer of 0.09 mm thick masking tape (Shurtape Technology, Inc: Hickory, N.C.) to the surfaces of the applicator that are in contact with the aluminum panel in order to minimize paint film defect inducing chatter during applicator motion. The resulting wet paint films were dried indoors for 7 days under ambient laboratory lighting conditions at a temperature of about 20° C. and a relative humidity of about 50%. Paint film dimensions after drying were as follows: 27.94 cm long, 7.62 cm wide, and between 0.06 mm and 0.11 mm thick. Paint film thicknesses were determined using a Dualscope FMP40C measuring device (Fischer Technologies Inc: Windsor, Conn.).

### Preparation B. Dirt Deposition

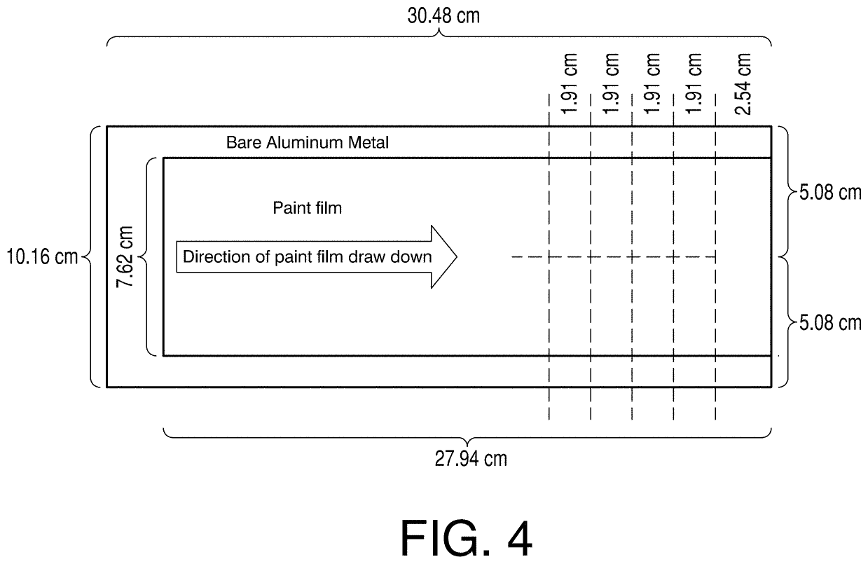

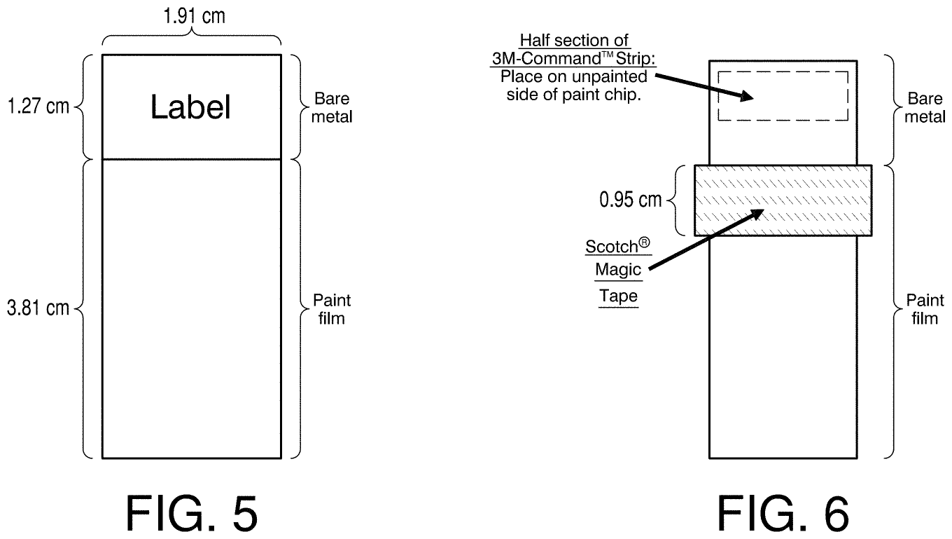

The paint film coated panels produced as described in Preparation A were cut into smaller pieces as shown in FIG. 4 using a 30.48 cm blade width, hand operated sheet metal cutter (Di-Acro: Oak Park Heights, Minn.) taking care not to damage the associated paint films. The bottom 10.16 cm long×2.54 cm wide section of each paint panel was discarded. The remaining 5.08 cm long×1.91 cm wide paint panel pieces, from this point forward referred to as chips, were labeled as shown in FIG. 5 using a standard permanent marker. A half-section of a 1.91 cm long×0.64 cm wide piece of a 3M Command™ picture frame hanging strip (3M Co; Maplewood, Minn.) and a 2.54 cm long×0.95 cm wide strip of Scotch® Magic™ tape (3M Co; Maplewood, Minn.) were then affixed, the latter with light pressure, to the unpainted and painted side of each chip, respectively, as shown in FIG. 6. The tape masked chips (typically twelve per evaluation) were then evenly attached (in randomized order) in circumferential fashion to the outer wall of the aerosolizing device of the present invention at its outlet end. Sections of 3M Command™ picture frame hanging strips (3M, Maplewood, Minn.) that had been previously placed on the outside of the outlet end of said device facilitated chip attachment. The orientation of the chips after their attachment to the aerosolizing device was such that efficient indirect contact of the chip paint film surfaces with the aerosolized particle stream exiting from said device occurs. The aerosolizing device included a modified eductor with venturi design.

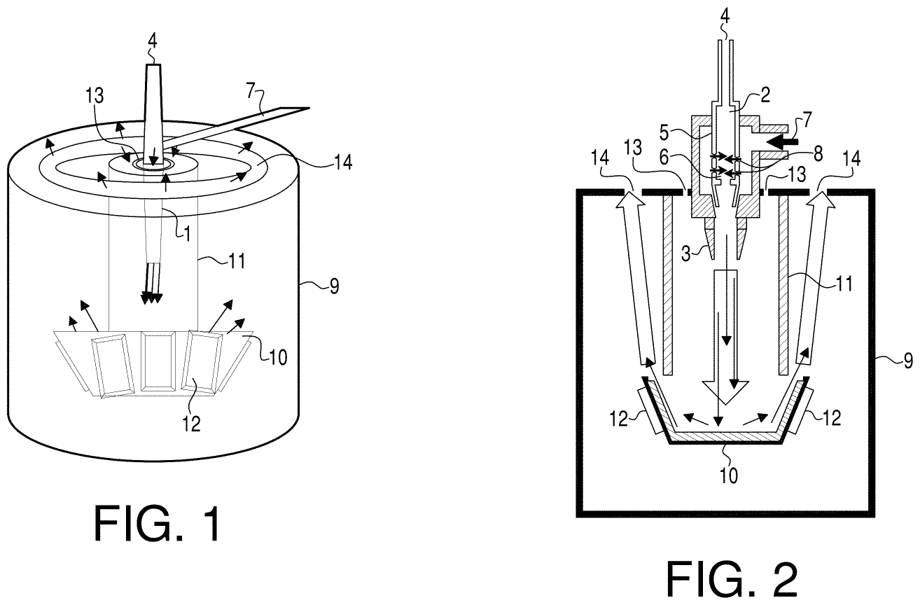

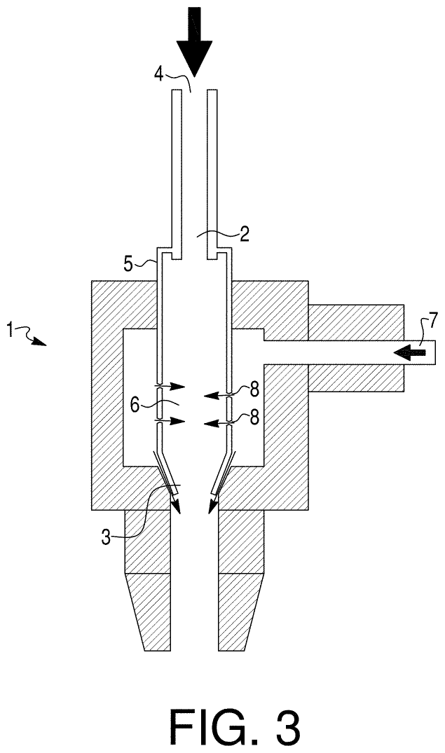

After placing the aerosolizing device according to FIG. 3 and the associated mounted chips into the enclosure of the present invention according to FIGS. 1-2, high pressure air (207 kPa, delivered using flexible tubing of 0.40 cm inner diameter) was introduced at a constant flow rate into the inlet end of said device. Taking advantage of the Venturi Effect caused by the air flow into and through the aerosolizing device, 50 mg portions of carbon black that ranged in primary particle size from about 100 nm to about 200 nm (Lamp Black 101 Powder unless otherwise specified) were sucked into the inlet end of said device through a 0.95 cm inner diameter, metal J-tube port. Each 50 mg portion of carbon black was added over about a minute and an additional minute was allowed to pass before the next portion was added. The number of carbon black portions run through the aerosolizing device was chosen, for example 3 to 30 portions, so as to yield the desired level of paint film darkening that resulted from the deposition onto said film of the now highly deagglomerated carbon black particles.

After the carbon black particle deposition process was completed, air was allowed to flow through the device for 5 more minutes, then the enclosure was opened, the aerosolizing device and the associated mounted chips removed from the enclosure interior, and the carbon black soiled chips carefully detached from said device. For each carbon black soiled chip, the strip of tape that was previously applied to each chip was then carefully removed yielding unsoiled paint film surface which was designated as Area 1.

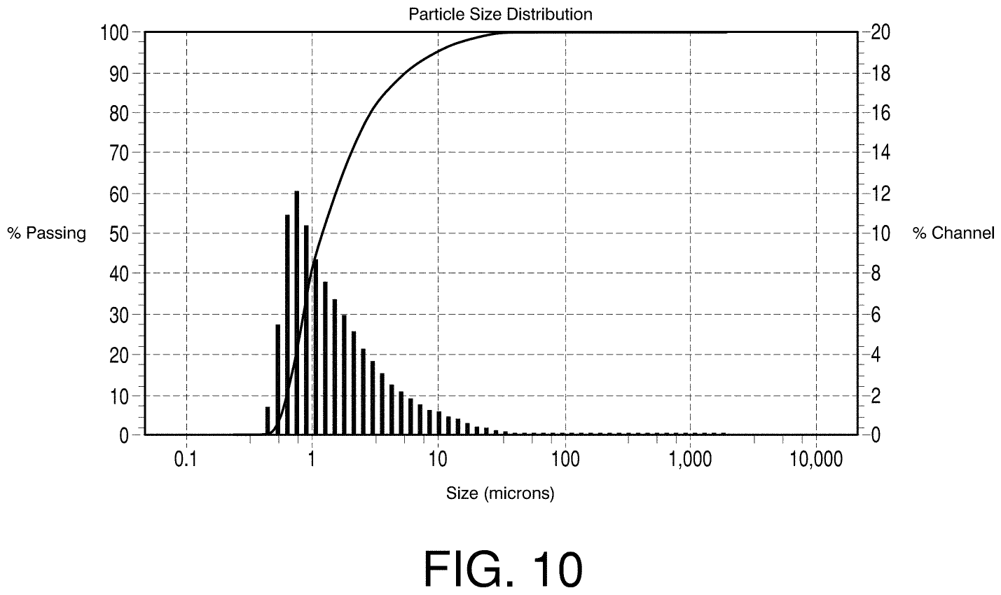

The size distribution of carbon black (Flamrus 101) was analyzed at the exit of the aerosolizing device eductor using a Microtrac S3500 laser diffraction particle analyzer. Briefly, 50 mg of sample was fed to said eductor using carbon black deposition conditions similar to those described in Preparation B (414 kPa feed pressure, eductor notch setting of 1) and the particle size distribution of the carbon black was obtained assuming irregular, absorbing particles. The particle sized distribution is provided in FIG. 10. Laser diffraction particle sizes are typically larger than mass median aerodynamic particle size. These data demonstrate that the aerosolizing device can yield particle size values below 2.5 microns in size in amounts greater than 50% by mass or volume.

### Preparation C. Moderate Single-Incubation Dirt Pickup Analysis

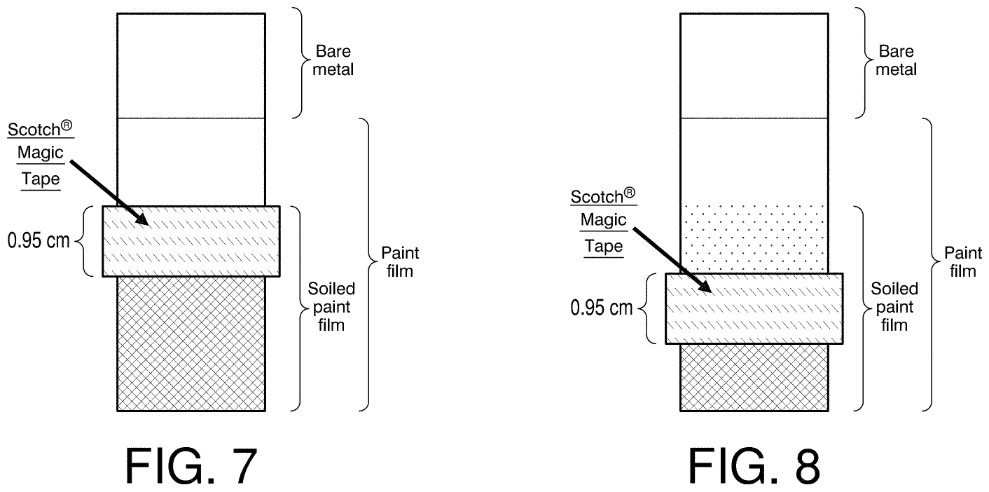

Following the process described in Preparation B, an additional 2.54 cm long×0.95 cm wide strip of Scotch® Magic™ tape was then affixed to the carbon black dusted side of each chip as shown in FIG. 7 using just enough pressure to ensure uniform contact of the tape adhesive with the soiled paint film surface. Said tape strip was then immediately and carefully removed from the chip, discarded, and the tape addition/tape removal process repeated. The paint film area affected by this tape peel process was designated as Area 2.

The carbon black treated chips were then placed flat into an aluminum pan (paint film side facing up) that was situated inside a standard, resistively heated, Blue M laboratory oven (General Signal, Blue Island, Ill.) that had been pre-heated to 45° C. After 72 hours of heating in air, said chips were removed from the oven and allowed to equilibrate to room temperature. An additional 2.54 cm long×0.95 cm wide strip of Scotch® Magic™ tape was then affixed to the carbon black dusted side of each chip as shown in FIG. 8 using just enough pressure to ensure uniform contact of the tape adhesive with the soiled paint film surface. Said tape strip was then immediately and carefully removed from the chip, discarded, and the tape addition/tape removal process repeated. The paint film area affected by this tape peel process was designated as Area 3.

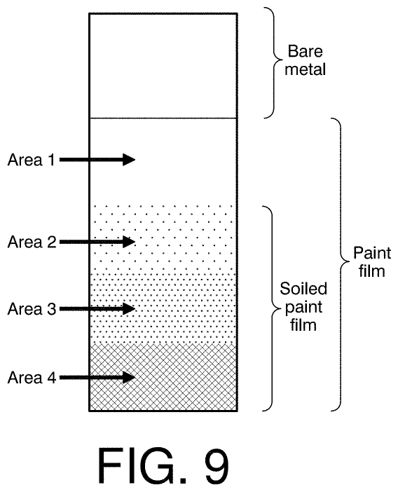

The remaining paint film area (carbon black soiled, oven heated, no tape peel) was designated as Area 4. A summary of the various chip areas identified above is provided in FIG. 9.

The carbon black dusted and heated chips were then very carefully placed onto the middle area of the glass exposure plate of a document scanner (Epson Perfection V750 PRO, Epson America: Long Beach, Calif.) paint film side down along with a white-gray-black striped, gray scale control card (X-rite: Grand Rapids, Mich.). (The entire area of the scanner exposure plate had been previously found to provide consistent image reproduction.) Using the scanner software (Epson Scan, Professional Mode), a tagged image file format (.tiff) based scan of the chips and control card was performed using 24 bit colour, 400 dot-per-inch resolution. The average grayscale values (0 to 255, 0=pure black and 255=pure white) of Areas 1 and 3 of each chip were determined using ImageJ image analysis software (National Institutes of Health: Bethesda, Md.). (Analysis of Area 2 indicates the presence of ambient temperature carbon black soiling; the chip-to-chip consistency of this data is used as a quality check to ensure operability of the dusting device and that deposition amounts on the paint chips are reasonably similar, e.g., within 20 greyscale units). The average grayscale value of the control card gray stripe was also determined and compared across multiple scanner runs to ensure scanner operation consistency. The average grayscale values determined for Areas 1 and 3 were used to calculate an average delta grayscale (ΔGrayscale) value for each chip using Equation (1):

Average ΔGrayscale=(Average grayscale value for Area 1)−(Average grayscale value for Area 3) Equation (1)

wherein Area 1 is the undusted paint film surface after oven heating and Area 3 is the carbon black dusted paint film surface after oven heating and subsequent double tape peel. Larger average ΔGrayscale values equate to greater carbon black pick-up by a paint film surface.

### Preparation D. Sequential Multiple-Incubation Dirt Pickup Analysis

A modification of Preparation C to include additional incubation and tape peel steps was applied to determine additional temperature dependent or time dependent dirt pickup properties of paint films. In Preparation C, Area 1 represents the undusted paint film surface after oven heating, Area 2 represents ambient room temperature dirt pickup and Area 3 represents dirt pickup after the identified temperature incubation. The location and size of Area 1, Area 2 and Area 3 can be varied. The remainder of the chip may also be designated Area 4 and represents mechanically undisturbed deposited carbon black after oven treatment.

In a multiple incubation, the paint chip was divided into n more areas where n represents the number of additional heat treatments and/or incubation time variances. For example, a five-step temperature incubation would have 5+3 areas. Five areas would be reserved for the five specified temperature steps and three would be reserved for Area 1, Area 2, and Area 4 as indicated in Preparation C. Additional Areas 3, 5, 6, 7 and 8 were assigned by the desired experimental protocol.

For example, the evaluation of the dirt pickup of the paint film at multiple incubation temperatures for equivalent incubation times in succession were performed. A suitable time may be 1 hr and suitable temperatures may be, 60° C., 80° C., 100° C., 120° C. Another typical experimental protocol is the evaluation of the time dependent dirt pickup of the paint film under isothermal conditions. A suitable temperature may be 45° C. or 60° C. for linearly or logarithmically spaced time intervals spanning minutes to days. After each specific incubation, the paint chip is removed from the oven, allowed to equilibrate to room temperature, and is then tape peeled as indicated in Preparation C.

For these experiments, the dirt pickup of the paint film was evaluated through changes in optical contrast, surface elemental composition or other factors impacted by the presence of dirt. As indicated by Equation (1), greyscale values were applied, as done with the use of carbon black in the present invention.

For any given experiment, dirt pickup of the paint film was determined for each respective Area using Equation (2):

Average ΔGrayscale=(Average grayscale value for Area 1)−(Average grayscale value for Area X) Equation (2)

where Area 1 is the Undusted paint film surface after oven heating and Area X is the carbon black dusted paint film surface after specified incubation and subsequent double tape peel.

### Preparation E. Mass Concentration of Solid Particles Having a Specified MMAD

The mass concentration of carbon black having a MMAD below 10 μm or below 2.5 μm, achieved by the aerosolization device, was determined using inertial impaction particle sampling collection devices. A PM10 Impact Sampler aerosol sampling collection device (Cat. No. 225-390; SKC Inc., Eighty Four, Pa.) and separately a PM2.5 Impact Sampler (Cat. No. 225-392; SKC Inc., Eighty Four, Pa.) were placed in housing 11 according to FIGS. 1-2 and Preparation B. Each Impact Sampler was loaded with a 47-mm Quartz filter (Tissuquartz 2500QAT-UP PALLFLEX Membrane filters; Pall Lifesciences, Port Washington, N.Y.) and a pre-oiled 37-mm impaction disc (Cat. No. 225-395, SKC Inc., Eighty Four, Pa.). Each quartz filter was weighed in quadruplicate on an analytical balance with a 0.01 mg resolution prior to insertion into the filter cassette assembly. The filter cassette assembly containing both the quartz filter and the impaction disc was also pre-weighed in quadruplicate. The device was assembled and the SKC Impact samplers were connected to an air sampling pump calibrated to operate at 10 L/min per manufacturer's instructions.

In each experiment, an impactor was placed at the bottom of housing 11 within 75 mm of the sample attachment positions. The chamber was closed and operated as usual. Air at 207 kPa was fed into the aerosolizing device at a constant flow rate. Three 50-mg portions of carbon black was fed into the metal J-tube port over the course of approximately 1 minute, taking advantage of the venturi effect with approximately one minute of wait time between each feeding. The air sampling pump was turned on 1 minute prior to the first particle feeding and turned off 1 minute after the three particle feedings were complete. The air velocity across the exit of the 33 mm circular exhaust port was determined to be 4.03 m/s using a hotwire anemometer (Amprobe TMA-21HW; Amprobe, Everett, Wash.). Operability of the anemometer was confirmed by measuring the air velocity at the inlet of a calibrated SKC Field Rotometer with a reported accuracy of 3% (Cat. No. 320-4A20L; SKC Inc., Eighty Four, Pa.). At a flowrate of 10 Lpm, an air velocity of 1.51 m/s was measured through a circular port of 8.5 mm in diameter. The volumetric flow rate through an orifice can be determined by product of the cross-sectional area of the orifice and air flow velocity. Accordingly, the volumetric air flow from the rotameter using the anemometer measurements and port diameter is 10.3 Lpm in agreement with the rotameter reading of 10 Lpm. The volumetric flow rate out of the aerosol chamber was likewise determined to be 414 Lpm.

After each sampling experiment, the aerosol chamber was disassembled and the aerosol sampling collection device was removed. The mass concentration was determined by measuring the total change in mass of the filter cassette assembly in quadruplicate. The PM10 or PM2.5 mass was determined by disassembling the filter housing, per manufacturers instruction, and weighing the filters in quadruplicate.

The results of the experiments and calculated PM10 and PM2.5 content are summarized in Tables 1 and 2. FIGS. 11B and 11C show that the carbon black is clearly primarily deposited on the quartz filter, as indicated by the color change as compared with FIG. 11A. This demonstrates a high percentage, or concentration, of MMAD below the aerosol sampling collection device threshold.

TABLE 1
Mass Concentration of Particles with MMAD Below 10 μm Using
SKC PM10 Impact Sampler

| | Total Mass of | | |
|---|---|---|---|
| | Particles | PM10 Mass | Percent MMAD |
| Solid Powder | Sampled (mg) | Collected (mg) | below 10 μm |
| Flamrus 101 | 5.06 ± 0.03 | 4.76 ± 0.05 | 94.1% ± 1.2% |
| Lamp Black 101 | 5.03 ± 0.05 | 4.90 ± 0.06 | 97.4% ± 1.6% |

TABLE 2
Mass Concentration of Particles with MMAD Below 2.5 μm Using
SKC PM2.5 Impact Sampler

| | Total Mass of | | |
|---|---|---|---|
| | Particles | PM2.5 Mass | Percent MMAD |
| Solid Powder | Sampled (mg) | Collected (mg) | below 2.5 μm |
| Flamrus 101 | 4.78 ± 0.06 | 4.44 ± 0.03 | 92.9% ± 1.4% |
| Lamp Black 101 | 5.47 ± 0.06 | 5.10 ± 0.06 | 93.3% ± 2.8% |

### EXAMPLES

### Comparative Example 1. Outdoor Exposure Testing in Guangzhou, China

Seven paint film coated panels, each derived from a different commercially available exterior paint formulation and prepared using the procedure described in Preparation A, were sent for outdoor exposure testing in Guangzhou, China. Outdoor exposure testing of the associated paint films was then initiated per ASTM test methods G147-2009 and G7-2013 and in accordance with the generally recognized governing standards for the outdoor evaluation of paint. The panels were mounted facing south at a 45 degree angle from horizontal and without any backing on a 359 cm long×164 cm wide aluminum exposure rack that was positioned over grassy groundcover. Starting at the zero hour exposure time point, spectral measurements (400 nm to 700 nm in 20 nm increments) of the paint films were performed at periodic intervals per ASTM test methods E1349-06 (2013) and E308-2013 using an X-Rite 948 reflection spectrocolorimeter (X-Rite, Inc: Grand Rapids, Mich.; D65 CIE standard illuminant, 0 degree illumination angle, 45 degree viewing angle). Each measurement consisted of gathering spectral reflectance data from three widely separated areas of a paint film and averaging the results to produce a corresponding HunterLab color scale based average L* value (white-black colour axis). Obtained average L* values were then used to calculate an average dirt pick-up value (average ΔL*) for each paint film at various exposure time points using Equation (3):

Average ΔL*=(0 hour exposure, average L*)−(X hour exposure, average L*). Equation (3)

Larger average ΔL* values equate to greater dirt pick-up. Table 3 summarizes the average ΔL* values obtained as a function of outdoor exposure time for each of the seven evaluated paint films.

TABLE 3
Average ΔL* for Comparative Example 1 Versus
Exposure Time in Days

| | | Shield- | | Super- | | | |
|---|---|---|---|---|---|---|---|
| | 7 in 1 | 1 Nano | Shield- | shield | Super- | | |
| | Future | Semi- | 1 Nano | Semi- | shield | Aquis | |
| Day | Color | Gloss | Sheen | Gloss | Sheen | Façade | Novasil |
| 0 | 0.00 | 0.00 | 0.00 | 0.00 | 0.00 | 0.00 | 0.00 |
| 56 | 10.66 | 8.26 | 1.88 | 8.30 | 1.95 | 2.39 | 2.51 |
| 95 | 13.07 | 9.79 | 3.30 | 10.05 | 3.01 | 3.19 | 3.34 |
| 140 | 13.93 | 11.36 | 4.14 | 11.46 | 3.96 | 4.39 | 4.89 |
| 172 | 13.76 | 11.98 | 4.74 | 11.04 | 4.69 | 4.42 | 4.82 |
| 236 | 18.11 | 15.35 | 10.19 | 15.13 | 8.68 | 10.84 | 10.50 |
| 272 | 19.22 | 16.23 | 10.64 | 14.83 | 9.66 | 11.38 | 11.19 |
| 302 | 19.28 | 17.02 | 10.89 | 15.35 | 10.14 | 12.2 | 12.06 |
| 333 | 21.41 | 16.18 | 12.07 | 15.58 | 10.73 | 13.34 | 12.85 |
| 363 | 21.15 | 16.53 | 12.09 | 16.04 | 10.43 | 13.42 | 13.56 |
| 394 | 21.41 | 16.64 | 12.29 | 15.44 | 10.73 | 12.83 | 13.35 |

### Comparative Example 2. Outdoor Exposure Testing in Chennai, India

A duplicate set of the seven paint film coated panels highlighted in Comparative Example 1 were prepared using the procedure described in Preparation A. These were for outdoor exposure site in Chennai, India. Outdoor exposure testing of the associated paint films was then initiated per ASTM test methods G147-2009 and G7-2013 and in accordance with the generally recognized governing standards for the outdoor evaluation of paint. The panels were mounted facing south at a 45 degree angle from horizontal and without any backing on a 359 cm long×164 cm wide aluminum exposure rack that was positioned over grassy groundcover. Starting at the zero hour exposure time point, spectral measurements (360 nm to 750 nm in 10 nm increments) of the paint films were performed at periodic intervals per ASTM test method E1331 using an X-Rite Color i7 spectrophotometer (X-Rite, Inc: Grand Rapids, Mich.; D65 CIE standard illuminant, 0 degree illumination angle, 10 degree viewing angle, specular reflectance excluded). Each measurement consisted of gathering spectral reflectance data from three widely separated areas of a paint film and averaging the results to produce per ASTM test method E308 a corresponding HunterLab color scale based average L* value (white-black colour axis). Obtained average L* values were then used to calculate an average dirt pick-up value (average ΔL*) for each paint film at various exposure time points using Equation (3). Table 4 summarizes the average ΔL* values obtained as a function of outdoor exposure time for each of the seven evaluated paint films.

TABLE 4
Average ΔL* for Comparative Example 2 Versus
Exposure Time in Days

| | | Shield- | | Super- | | | |
|---|---|---|---|---|---|---|---|
| | 7 in 1 | 1 Nano | Shield- | shield | Super- | | |
| | Future | Semi- | 1 Nano | Semi- | shield | Aquis | |
| Day | Color | Gloss | Sheen | Gloss | Sheen | Façade | Novasil |
| 0 | 0.00 | 0.00 | 0.00 | 0.00 | 0.00 | 0.00 | 0.00 |
| 33 | 5.46 | 4.54 | 1.69 | 4.50 | 1.32 | 1.77 | 1.29 |
| 112 | 11.75 | 9.33 | 4.57 | 8.02 | 3.45 | 4.65 | 4.26 |
| 138 | 12.32 | 9.95 | 5.69 | 8.75 | 4.61 | 5.86 | 5.51 |
| 156 | 13.57 | 10.36 | 6.51 | 8.81 | 5.40 | 6.76 | 6.45 |
| 182 | 14.43 | 11.02 | 7.98 | 8.93 | 6.61 | 8.46 | 7.93 |
| 241 | 15.78 | 11.16 | 8.63 | 8.88 | 7.13 | 9.28 | 9.43 |
| 272 | 16.75 | 11.54 | 9.68 | 9.05 | 7.92 | 10.24 | 10.18 |
| 363 | 17.61 | 11.89 | 10.13 | 9.27 | 8.20 | 10.50 | 10.70 |

### Example 1. Accelerated Testing of Commercial Paints

An additional duplicate set of the seven paint film coated panels highlighted in Comparative Example 1 were prepared using Preparation A. Using the procedures described in Preparations B and C, said panels were then cut into chips, two of which for each paint type were then simultaneously evaluated for their accelerated dirt pick-up resistance. A total of 6 sequentially run carbon black (Flamrus 101) dusting passes were employed during said evaluation. The average ΔGrayscale values derived from each paint type chip pair are provided in Table 5.

TABLE 5
Average ΔGrayscale Values of Example 1

| | Shield-1 | | Super- | | | |
|---|---|---|---|---|---|---|
| 7 in 1 | Nano | Shield-1 | shield | Super- | | |
| Future | Semi- | Nano | Semi- | shield | Aquis | |
| Color | Gloss | Sheen | Gloss | Sheen | Façade | Novasil |
| 26.6 | 7.2 | 1 | 4.6 | 0.8 | 3.4 | 3.9 |

For each of the Guangzhou test site non-zero outdoor exposure times highlighted in Table 3 (ten in total), the ΔL* value of each listed paint was plotted against the corresponding ΔGrayscale value provided in Table 5. A linear least squares data fit was then performed for each of the ten plots. The resulting correlation coefficients (R² values) are provided in Table 6.

For each of the Chennai test site non-zero outdoor exposure times highlighted in Table 4 (eight in total), the ΔL* value of each listed paint was also plotted against the corresponding ΔGrayscale value provided in Table 5. A linear least squares data fit was performed for each of the eight plots. The resulting correlation coefficients are provided in Table 7.

TABLE 6
Correlation Coefficient of Example 1 with Comparative
Example 1

| Days of | Correlation |
|---|---|
| Exposure | Coefficient (R²) |
| 56 | 0.60 |
| 95 | 0.63 |
| 140 | 0.59 |
| 172 | 0.57 |
| 236 | 0.67 |
| 272 | 0.75 |
| 302 | 0.71 |
| 333 | 0.88 |
| 363 | 0.83 |
| 394 | 0.87 |

TABLE 7
Correlation Coefficient of Example 1 with Comparative
Example 2

| Days of | Correlation |
|---|---|
| Exposure | Coefficient (R²) |
| 33 | 0.56 |
| 112 | 0.72 |
| 138 | 0.74 |
| 156 | 0.84 |
| 182 | 0.90 |
| 241 | 0.94 |
| 272 | 0.93 |
| 363 | 0.93 |

The data provided in Tables 6 and 7 show that the correlation between the ΔL* values derived from outdoor exposure and the ΔGrayscale values derived from the current invention for a given series of paint films improves with increasing paint film outdoor exposure time ultimately allowing the latter value (for a given paint) to usefully predict the former beginning at about the 6 month to about the 12 month outdoor exposure time point depending on exposure site.

### Example 2. Accelerated Testing of Paints with Polymeric Binders of Varied T_{g} Values

Methyl methacrylate (MMA), methacrylic acid (MAA), and butyl acrylate (BA) monomers were utilized in differing amounts to prepare four unique polymeric binders as aqueous emulsions using emulsion polymerization techniques known to those skilled in the art. The amounts of each monomer used for each polymeric binder synthesis (mmol basis) are shown in Table 8. Also shown in Table 8 are the weight % solids of each produced emulsion and the glass transition temperature (T_{g}) of the corresponding polymeric binder. Emulsion weight % solids were determined gravimetrically by drying an emulsion sample for 2 hours at 110° C. in a standard vacuum oven. The glass transition temperature of a dried, solid sample of a polymeric binder was measured using a TA Instruments Q100 differential scanning calorimeter (TA Instruments, New Castle, Del.) and associated software.

TABLE 8
Composition and Characterization of Polymeric Binders 1-4

| | | | | Mole | Emulsion | Polymeric |
|---|---|---|---|---|---|---|
| | MMA | MAA | BA | Ratio | Wt % | Binder T_{g} |
| | (Mmol) | (MMol) | (MMol) | MMA/BA | Solids | (° C.) |
| 1 | 150 | 7 | 50 | 75/25 | 46.8 | 61 |
| 2 | 120 | 7 | 80 | 60/40 | 46.9 | 30 |
| 3 | 80 | 7 | 120 | 40/60 | 47.2 | 0 |
| 4 | 50 | 7 | 150 | 25/75 | 47.3 | −18 |

The four synthesized polymeric binders were each separately incorporated as their corresponding aqueous emulsions into the high quality test paint formulation described in Table 9 using paint manufacturing techniques that are known to those skilled in the art.

TABLE 9
Paint Composition of Example 2

| Binder | 1 | 2 | 3 | 4 |
|---|---|---|---|---|
| | Volume % | | | |
| Grind base: | | | | |
| HEC Thickener | 12.00 | 12.00 | 12.00 | 12.00 |
| Water | 4.52 | 4.52 | 4.52 | 4.52 |
| Dispersant | 2.00 | 2.00 | 2.00 | 2.00 |
| Surfactant | 0.25 | 0.25 | 0.25 | 0.25 |
| Defoamer | 0.14 | 0.14 | 0.14 | 0.14 |
| Biocide | 0.21 | 0.21 | 0.21 | 0.21 |
| TiO₂ | 6.00 | 6.00 | 6.00 | 6.00 |
| Calcined Clay | 8.01 | 8.01 | 8.01 | 8.01 |
| Diatomaceous Earth | 0.71 | 0.71 | 0.71 | 0.71 |
| Letdown: | | | | |
| Water | 4.52 | 4.52 | 4.52 | 4.52 |
| Polymeric Binder | 45.94 | 45.85 | 45.55 | 45.46 |
| (Aqueous Emulsion) | | | | |
| Opaque Polymer | 5.75 | 5.75 | 5.75 | 5.75 |
| Defoamer | 0.21 | 0.21 | 0.21 | 0.21 |
| Texanol | 1.25 | 1.00 | 0.75 | 0.44 |
| Propylene glycol | 0.70 | 0.93 | 1.16 | 1.48 |
| High Shear Rate HEUR | 2.00 | 2.00 | 2.00 | 2.00 |
| Thickener (20 wt % solids) | | | | |
| Adjustment: | | | | |
| Water | 5.88 | 6.00 | 6.24 | 6.35 |
| Total: | 100.1 | 100.1 | 100.0 | 100.1 |
| Pigment Volume | 46.46 | 46.46 | 46.46 | 46.46 |
| Concentration (%): | | | | |
| TiO₂ Content (kg/L of paint): | 0.24 | 0.24 | 0.24 | 0.24 |
| Paint Volume % Solids: | 37.37 | 37.37 | 37.39 | 37.38 |
| Paint Weight % Solids: | 52.66 | 52.65 | 52.68 | 52.66 |

Two duplicate sets of four paint film coated panels derived from the four produced test paints were prepared using the procedure described in Preparation A. Using the procedures of Preparations B and C, one set of panels was then cut into chips two of which for each test paint were then simultaneously evaluated for their accelerated dirt pick-up resistance. A total of 24 sequentially run carbon black dusting passes were employed during said evaluation. The average ΔGrayscale values derived from each test paint chip pair are provided in Table 10.

The remaining set of four (uncut) panels were sent to an industrial site located in Kuan Yin, Taiwan, where they were exposed outdoors. The panels were mounted facing south at a 90 degree angle from horizontal and without any backing on a 359 cm long×164 cm wide aluminum exposure rack that was positioned on a concrete base. Starting at the zero hour exposure time point, spectral measurements (360 nm to 750 nm in 10 nm increments) of the paint films were performed at periodic intervals per ASTM test method E1331 using an X-Rite RM200QC handheld color analyzer (X-Rite, Inc: Grand Rapids, Mich.; D65 CIE standard illuminant, 0 degree illumination angle, 10 degree viewing angle, specular reflectance excluded). Each measurement consisted of gathering spectral reflectance data from three widely separated areas of a paint film and averaging the results to produce per ASTM test method E308 a corresponding HunterLab color scale based average L* value (white-black colour axis). Obtained average L* values were then used to calculate an average dirt pick-up value (average ΔL*) for each test paint film at various exposure time points using Equation (3). The average ΔL* value obtained at the 204 day exposure time point for each test paint is provided in Table 10.

TABLE 10
Comparison of ΔGrayscale with ΔL* of Example 2 Paints

| | Polymeric | | Accelerated | 204 Days Outdoor |
|---|---|---|---|---|
| | Binder | Polymeric | Test | Exposure at |
| | (Aqueous | Binder T_{g} | Average | Kuan Yin |
| Paint | Emulsion) | (° C.) | ΔGrayscale | Average ΔL* |
| A | 1 | 61 | 1.7 | 4.8 |
| B | 2 | 30 | 8.4 | 9.06 |
| C | 3 | 0 | 43.7 | 12.47 |
| D | 4 | −18 | 57.9 | 14.55 |

The data provided in Table 10 reveal the expected trend of increasing ΔL* and ΔGrayscale values (increasing dirt pick-up) with decreasing polymer binder glass transition temperature (decreasing paint film hardness). More importantly, a linear least squares data fit of a plot of the average ΔL* values shown in Table 10 versus their corresponding ΔGrayscale values (also shown in Table 10) yielded a correlation coefficient of 0.90, a value whose magnitude demonstrates that the ΔGrayscale values derived from the current invention can predict the outdoor exposure derived ΔL* values of a series of similar paints that possess polymeric binders of differing glass transition temperatures at a usefully long exposure time of 204 days.

### Example 3. Accelerated Testing of Paints with Different Pigment Volume Concentrations

Five paints with different pigment volume concentrations (PVC) were produced according to the recipe provided in Table 11 using paint manufacturing techniques that are known to those skilled in the art.

TABLE 11
Composition of Paints Having Differing PVC Values

| Paint | A | B | C | D | E |
|---|---|---|---|---|---|
| | | | | Flat | Flat |
| Paint | | Semi- | | (low | (high |
| Description | Glossy | gloss | Satin | PVC) | PVC) |
| | Volume % | | | | |
| Grind: | | | | | |
| Natrosol 250 | 0.00 | 0.00 | 0.00 | 12.00 | 12.00 |
| MHR (2.50 wt % | | | | | |
| Aqueous Solution) | | | | | |
| Water | 6.59 | 6.59 | 8.15 | 4.52 | 22.66 |
| Tamol 165A | 0.68 | 0.68 | 0.64 | 2.00 | 3.30 |
| (21 wt % Aqueous | | | | | |
| Solution) | | | | | |
| BYK-348 | 0.23 | 0.23 | 0.00 | 0.25 | 0.25 |
| Foamstar ST2434 | 0.14 | 0.14 | 0.14 | 0.14 | 0.14 |
| Kathon LX | 0.21 | 0.21 | 0.21 | 0.21 | 0.21 |
| (1.50 wt % Aqueous | | | | | |
| Solution) | | | | | |
| TiO₂ (Ti-Pure ™ | 6.00 | 6.00 | 6.13 | 6.00 | 0.00 |
| R-706) | | | | | |
| TiO₂ (Ti-Pure ™ Select | 0.00 | 0.00 | 0.00 | 0.00 | 6.39 |
| TS-6300) | | | | | |
| Minex 4 | 0.00 | 0.34 | 2.29 | 8.01 | 12.41 |
| Diafil 525 | 0.00 | 0.00 | 0.00 | 0.71 | 1.43 |
| Grind subtotal: | 13.85 | 14.20 | 17.56 | 33.84 | 58.79 |
| Water | 5.40 | 5.40 | 0.00 | 0.00 | |
| Letdown: | | | | | |
| Water | 0.00 | 0.00 | 6.00 | 4.52 | 0.00 |
| Rhoplex VSR | 63.90 | 52.09 | 54.16 | 43.00 | 28.70 |
| 1049 LOE | | | | | |
| (50 wt % Aqueous | | | | | |
| Emulsion) | | | | | |
| Rhopaque Ultra | 4.50 | 4.50 | 5.81 | 5.75 | 2.10 |
| (30 wt % Aqueous | | | | | |
| Dispersion) | | | | | |
| BYK-348 | 0.00 | 0.00 | 0.20 | 0.00 | 0.00 |
| Texanol | 0.70 | 0.56 | 0.58 | 0.50 | 0.50 |
| Foamstar ST2434 | 0.14 | 0.14 | 0.00 | 0.21 | 0.20 |
| Propylene glycol | 1.05 | 1.05 | 1.26 | 1.42 | 1.40 |
| Ammonia | 0.10 | 0.10 | 0.05 | 0.00 | 0.00 |
| (28 wt % Aqueous | | | | | |
| Solution) | | | | | |
| Acrysol RM2020 NPR | 2.40 | 3.77 | 2.97 | 2.00 | 2.90 |
| (20 wt % Aqueous | | | | | |
| Dispersion) | | | | | |
| Acrysol SCT-275 | 0.23 | 0.57 | 0.25 | 0.00 | 0.00 |
| (17.5 wt% Aqueous | | | | | |
| Dispersion) | | | | | |
| Adjustment: | | | | | |
| Water | 7.84 | 17.67 | 11.15 | 8.81 | 5.40 |
| Total: | 100.1 | 100.0 | 100.0 | 100.1 | 100.0 |
| Pigment Volume | 21.5 | 26.0 | 30.8 | 46.5 | 60.7 |
| Concentration (%): | | | | | |
| TiO₂ Content (kg/L | 0.24 | 0.24 | 0.24 | 0.24 | 0.24 |
| of paint): | | | | | |
| Volume % Solids | 37.57 | 32.53 | 36.25 | 37.38 | 33.49 |
| Weight % Solids | 48.47 | 44.10 | 48.54 | 52.70 | 52.89 |
| Paint Density (kg/L) | 1.21 | 1.21 | 1.25 | 1.34 | 1.41 |

Two duplicate sets of five paint film coated panels derived from the five produced test paints were prepared using the procedure described in Preparation A. Using the procedures described in Preparations B and C, one set of panels was then cut into chips two of which for each test paint were then simultaneously evaluated for their accelerated dirt pick-up resistance. A total of 24 sequentially run carbon black dusting passes were employed during said evaluation. The average ΔGrayscale values derived from each paint chip pair are provided in Table 12.

The remaining set of five (uncut) panels were sent to an industrial site located in Kuan Yin, Taiwan, where they were exposed outdoors and analyzed as described in Example 2. The average ΔL* value obtained at the 204 day exposure time point for each test paint is provided in Table 12.

TABLE 12
Comparison of ΔGrayscale with ΔL* of Example 3 Paints:

| | | | | 204 Days |
|---|---|---|---|---|
| | | | Accelerated | Outdoor |
| | | Paint | Test | Exposure at |
| | Paint | Formulation | Average | Kuan Yin |
| Paint | Type | PVC | ΔGrayscale | Average ΔL* |
| A | Glossy | 21.5 | 82.7 | 14.78 |
| B | Semi- | 26.0 | 77.5 | 13.78 |
| | Gloss | | | |
| C | Satin | 30.8 | 73.4 | 13.44 |
| D | Flat | 46.5 | 38.3 | 10.61 |
| E | Flat | 60.7 | 7.4 | 9.23 |

The data provided in Table 12 reveal the expected trend of decreasing ΔL* and ΔGrayscale values (decreasing dirt pick-up) with increasing paint pigment volume concentration (increasing paint inorganic content). Additionally, a linear least squares data fit of a plot of the average ΔL* values shown in Table 12 versus their corresponding ΔGrayscale values (also shown in Table 12) yielded a correlation coefficient of 0.97, a value whose magnitude demonstrates that the ΔGrayscale values derived from the current invention can predict the outdoor exposure derived ΔL* values of a series of similar paints that possess differing pigment volume concentrations at a usefully long exposure time of 204 days.

### Example 4. Effect of Temperature Exposure on Test Samples

Paint chips obtained from the same panels prepared for Comparative Example 2 were dusted with carbon black (Flamrus 101) following the procedure in Preparation B except that only 6 passes were applied. Said chips were then subjected to a sequential multiple-treatment dirt pickup analysis. Four treatments were chosen as follows: 60° C. for 1 hour, 80° C. for 1 hour, 100° C. for 1 hour, and 120° C. for 1 hour. After each treatment, the chips were allowed to equilibrate to room temperature. A double tape peel was then performed at a designated area after which the chips were returned to the oven for subsequent treatments in accordance with Preparation D, and ΔGrayscale values were measured (Table 13).

TABLE 13
ΔGrayscale Values of Samples at Different Temperature
Exposures

| | | Shield- | | Super- | | | |
|---|---|---|---|---|---|---|---|
| | 7 in 1 | 1 Nano | Shield- | shield | Super- | | |
| Temp | Future | Semi- | 1 Nano | Semi- | shield | Aquis | |
| (° C.) | Color | Gloss | Sheen | Gloss | Sheen | Façade | Novasil |
| 60 | 24 | 3 | 0 | 5 | 0 | 0 | 5 |
| 80 | 35 | 4 | 0 | 9 | 4 | 1 | 3 |
| 100 | 60 | 9 | 3 | 14 | 5 | 2 | 5 |
| 120 | 71 | 18 | 6 | 16 | 8 | 4 | 5 |

The data in Table 13 demonstrates an additional approach for paint film characterization. Sequential incubations provide information indicative of thermal behavior of the paint surface films and also provide an alternative route to reasonable correlations with the outdoor data given in Tables 3 and 4.

### Comparative Example 3. Accelerated Test Using Particle Slurry

Paint film panels were prepared by applying paint to a film panel by brush, allowing the sample to dry. Slurries containing 25 wt % carbon black were made by mixing carbon black (10 g, Flamrus 101) in deionized water (30 g) sonicating the mixture for 4 minutes at 50% amplitude in a Qsonica (Newtown, Conn.) Q700 ultrasonic processor equipped with a ½ inch replaceable tip horn. The resulting slurry was cooled to room temperature and then applied by brush to cover ⅓ of the paint film panels to create the slurry treated area. The slurry treated panels were dried for 4 hours under laboratory conditions, rinsed with tap water and lightly wiped with a wet sponge. This process was conducted in a manner to prevent contamination and discoloring of a non-treated original paint controlled area of the paint film that was not brushed. This untreated area of the paint film is designated Area 1 and the slurry-treated area is designated Area 3 for Average ΔGrayscale. The rinsed and wiped panels were further air dried for another 24 hours before being carefully placed onto the middle area of the glass exposure plate of a document scanner (Epson Perfection V750 PRO, Epson America: Long Beach, Calif.) paint film side down along with a white-gray-black striped, gray scale control card (X-rite: Grand Rapids, Mich.). Using the scanner software (Epson Scan, Professional Mode), a tagged image file format (.tiff) based scan of the chips and control card was performed using 24 bit colour, 400 dot-per-inch resolution. The average grayscale values (0 to 255, 0=pure black and 255=pure white) of the control area and the brushed area was determined using ImageJ image analysis software (National Institutes of Health: Bethesda, Md.). The Average ΔGrayscale value for each panel was calculated using Equation (1) above, shown in Table 14. Larger average ΔGrayscale values equate to greater carbon black pick-up by a paint film surface.

TABLE 14
Average ΔGrayscale Values of Comparative Example 3

| 7 in 1 | Shield-1 | | | |
|---|---|---|---|---|
| Future | Nano Semi- | Shield-1 | Super-shield | Super-shield |
| Color | Gloss | Nano Sheen | Semi-Gloss | Sheen |
| 3.7 | 6.5 | 24.5 | 5.2 | 35.8 |

For each of the Chennai test site non-zero outdoor exposure times highlighted in Table 4 (eight in total), the ΔL* value of each listed paint was plotted against the corresponding ΔGrayscale value provided in Table 14. A linear least squares data fit was performed for each of the eight plots. The resulting correlation coefficients are provided in Table 15.

TABLE 15
Correlation Coefficient of Comparative Examples 2 and 3

| Days of | Correlation |
|---|---|
| Exposure | Coefficient (R²) |
| 33 | 0.93 |
| 112 | 0.85 |
| 138 | 0.84 |
| 156 | 0.74 |
| 182 | 0.62 |
| 241 | 0.50 |
| 272 | 0.41 |
| 363 | 0.38 |

The correlation between the ΔL* values derived from outdoor exposure and the ΔGrayscale values derived from the slurry treatment dirt pick-up assessment method show that the particle slurry examples are not predictors of outdoor performance because the correlation coefficient worsens with time.

### Comparative Example 4. Accelerated Test Without Thermal Treatment

Example 1 was repeated, except the samples were not heated in an oven for an incubation period. Applicants found no observable correlation between the visual interpretation of deposited carbon black and the results from outdoor exposures provided in Comparative Examples 1 and 2.

TABLE 16
Average ΔGrayscale Values of Comparative Example 4

| | Shield-1 | | Super- | | | |
|---|---|---|---|---|---|---|
| 7 in 1 | Nano | Shield-1 | shield | Super- | | |
| Future | Semi- | Nano | Semi- | shield | Aquis | |
| Color | Gloss | Sheen | Gloss | Sheen | Façade | Novasil |
| 0.0 | 0.0 | 0.0 | 0.0 | 0.0 | 0.0 | 0.0 |

### Comparative Example 5. Accelerated Test With Water Immersion

Comparative Example 4 was repeated, where the samples were not heated in an oven for an incubation period. After carbon black deposition, samples were immersed in water at pH 3 or deionized water (DI). The samples were allowed to dry for 24 hours, and loosely adhered dirt was removed by two tape peels as in previous experiments. The sample areas where the sample was not exposed to liquid were compared with areas treated with liquid and tape peel. Applicants found no observable correlation between the visual interpretation of deposited carbon black and the results from outdoor exposures provided in Comparative Examples 1 and 2.

TABLE 17
Average ΔGrayscale Values of Comparative Example 5

| | | Shield- | | Super- | | | |
|---|---|---|---|---|---|---|---|
| | 7 in 1 | 1 Nano | Shield- | shield | Super- | | |
| | Future | Semi- | 1 Nano | Semi- | shield | Aquis | |
| Water | Color | Gloss | Sheen | Gloss | Sheen | Façade | Novasil |
| pH 3 | 2.6 | 1.8 | 2.1 | 2.2 | 0.2 | 8.2 | 7.2 |
| DI | 0.8 | 0.0 | 0.5 | 0.0 | 0.0 | 2.5 | 2.8 |

What is claimed is:

1. A process for quantifying solid residue on a sample comprising:
1) providing at least one solid substrate and an aerosolizing device having an inlet and an outlet,
2) adding a solid material to the inlet,
3) forming a particle cloud of solid particles, wherein at least 1% of the mass concentration of solid particles have a mass median aerodynamic particle diameter up to about 10 μm, the particle cloud of solid particles exiting the aerosolizing device through the outlet, thus applying said solid particles to said at least one solid substrate to form at least one treated substrate,
4) wherein said at least one treated substrate is maintained at a temperature of from about 30 to about 120° C. for at least a portion of the process,
5) removing a portion of said solid particles from said at least one treated substrate, where steps 4) and 5) are performed in any order to form at least one sample, and
6) analyzing said at least one sample.

2. The process of claim 1, where at least 1% of the mass concentration of solid particles have a mass median aerodynamic particle diameter up to about 2.5 μm.

3. The process of claim 1, where the step of removing a portion of said solid particles is performed by contacting the sample with an adhesive tape or tacky surface and removing the tape or tacky surface, contacting with and removing a silicone film, applying vacuum, mechanical wiping, liquid washing, rubbing, or the use of a liquid or air jet.

4. The process of claim 1, where the step of providing at least one solid substrate is performed by positioning at least one solid substrate to avoid direct contact with the outlet of the aerosolizing device and allowing the particle cloud to contact said at least one solid substrate.

5. The process of claim 1, further comprising a step of applying electrostatic energy, thermophoretics, field focusing, rotational force, high speed mixing, continuous drop, pressure change, or aerodynamic enclosure design.

6. The process of claim 1, where said at least one sample is analyzed in step 6) for weight, brightness, color, reflectance, or chemical composition.

7. The process of claim 1, where the solid material is carbon black, iron oxide, graphite, ash, soot, crushed brick dust, dirt, pollen, spores, inorganic crystallites, or mixtures thereof.

8. The process of claim 1, where the step of adding a solid material to the inlet further comprises adding a carrier gas.

9. The process of claim 1, where said at least one solid substrate is polymeric, wood, wood laminate, paper laminate, or a solid surface having a coating, wherein the coating is a polymer coating, non-polymeric organic coating, or inorganic coating.

10. The process of claim 1, where the treated substrate is heated by oven or other controlled elevated temperature environment; heating an enclosure containing the treated substrates and the aerosolizing device; absorption of light; convective heating; conductive heating; or applying directed heat.

11. The process of claim 9, where said at least one substrate is pretreated before step 1).

12. The process of claim 1, where said at least one treated substrate is maintained at a temperature of from about 30 to about 120° C. for 5 minutes to 1 month before analysis.

13. An apparatus comprising:
a) an enclosure,
b) an aerosolizing device comprising a lumen extended from an inlet at one end to an outlet at another end, wherein the lumen is in fluid communication with the enclosure, and wherein the lumen allows an aerosol stream comprising gas and solid material to flow through the aerosolizing device and to exit the outlet of the aerosolizing device,
c) a port on the enclosure for adding solid material to the aerosolizing device, and
d) at least one solid substrate located in the enclosure,
wherein the aerosolizing device further comprises:
a particle dispersion unit for reducing agglomerates and/or aggregates to solid particles wherein at least 1% of the mass concentration of solid particles have a mass median aerodynamic particle diameter up to about 10 μm,
wherein said at least one solid substrate is located inside the enclosure and positioned to avoid direct contact with the aerosol stream exiting the outlet of the aerosolizing device.

14. The apparatus of claim 13, where the particle dispersion unit reduces agglomerates and/or aggregates to solid particles wherein at least 1% of the mass concentration of solid particles have a mass median aerodynamic particle diameter up to about 2.5 μm.

15. The apparatus of claim 13, where the aerosolizing device forces the aerosol stream through the lumen at a velocity up to about 50 m/s.

16. The apparatus of claim 13, where the aerosolizing device contains an intake for gas leading to a chamber, where the chamber connects to the particle dispersion unit at one or more ports allowing the gas to contact the solid particles.

17. The apparatus of claim 13, where said at least one solid substrate is polymeric, wood, wood laminate, paper laminate, or a solid surface having a coating, wherein the coating is a polymer coating, non-polymeric organic coating, or inorganic coating.

18. The apparatus of claim 13, further comprising a flow diverter inside the enclosure, where the flow diverter is positioned in the path of the aerosol stream exiting the aerosol device to divert the aerosol stream away from said at least one solid substrate.

19. The apparatus of claim 13, wherein the outlet of the aerosolizing device extends into the enclosure.

20. The apparatus of claim 13, further comprising one or more openings on the enclosure that connects the contents of the enclosure to atmospheric pressure, vacuum, a pressurized area, or a means for recirculating solid material.
